(19) United States
(12) Patent Application Publication
(10) Pub. No.: US 2022/0411355 A1
(43) Pub. Date: Dec. 29, 2022

(54) A SYSTEM FOR MONITORING, EVALUATING AND PROVIDING FEEDBACK OF PHYSICAL MOVEMENTS OF A USER

(71) Applicant: PreCure ApS, Allinge (DK)

(72) Inventors: Søren WÜRTZ, Allinge (DK); Andersen FINN BECH, Allinge (DK); Jønne MARCHER, Allinge (DK); Carsten SCHEIBYE, Allinge (DK)

(21) Appl. No.: 17/632,915

(22) PCT Filed: Aug. 6, 2020
(86) PCT No.: PCT/DK2020/050229

§ 371 Date: Feb. 4, 2022

(30) Foreign Application Priority Data
Aug. 6, 2019 (DK) PA 2019 70499

(51) Int. Cl. C07C 29/84

(52) U.S. Cl. CPC C07C 29/84

(57) ABSTRACT

The present invention relates to a system for monitoring a physical movements of a user, the system comprising: a detection unit configured for receiving a signal obtained by a sensor device representative of a sensed bodily activity, calculating a load index value and communicate, e.g. to the user wearing the sensor device, the value of the load index value. The invention also relates to a method for monitoring and evaluating physical movement of a user and providing feedback to the user.

### FIELD OF THE INVENTION

The present invention relates to a system for monitoring and preferably evaluating physical movements of a user, the system comprising: a detection unit configured for receiving a signal obtained by a sensor device representative of a sensed bodily activity, such as a muscle activity, calculating a load index value and communicate, e.g. to the user wearing the said sensor device, the value of the load index value. The invention also relates to a method for monitoring and evaluating physical movement of a user and providing feedback to the user.

### BACKGROUND OF THE INVENTION

At present, most prevention and rehabilitation efforts associated with muscular damage, the tendons and their origins and insertions—known as Musculoskeletal Disorder (MSD)—are addressed manually by instructing a person in the correct use of his/her muscular function. This procedure is expensive, time consuming and has limited lasting effect. The also other bodily injuries, such as ligament damage etc.

The few technical solutions, which exist, are characterised by being equipment-intensive and requires a professional handling the setup and use. This accounts for instance patent US2012071732, where the use is restricted by data being transferred by wire. The same patent is also limited in its use as it only enables measurement and not evaluation. Likewise, SEMG is today primarily used to measure, leaving diagnostics and movement analyses to professionals. This accounts for instances the patents US2016100775 and US2015070270.

There has been conducted significant amount of research relating to the prevention of strain and muscular damage, which has resulted in strain-level and time requirements for the prevention of strain damage for many different muscular and bodily groups and body positions. However, there has been little development and success translating these scientific findings into parameters and devices for the prevention and rehabilitation of these damages.

Hence, an improved method to monitor and evaluate muscular activity and provide feedback, eq. to the user, would be advantageous. This may be used in an attempt e.g. to reduce the impact and frequency of MSD which would be advantageous, and in particular a more efficient and/or reliable way to create lasting changes in the users' behaviour would be advantageous.

### OBJECT OF THE INVENTION

It is a further object of the present invention to provide an alternative to the prior art.

In particular, it may be seen as a preferred object of the present invention to provide a method to reducing the impact and frequency of MSD that solves the above mentioned problems of the prior art by creating lasting changes in users' behaviour through biofeedback, such as providing feedback based on the muscular activity.

### SUMMARY OF THE INVENTION

Thus, the above described object and several other objects are intended to be obtained in a first aspect of the invention by providing an system for monitoring and, preferably evaluating physical movements of a user, the system comprising:
a detection unit configured for
   receiving a signal obtained by a sensor device representative of a sensed bodily activity, such as a muscular activity, such as a position or movement of muscles, bone(s) and/or joint(s),
   calculate based on the signal obtained by the sensor device over a period of time, such as an 1 hour period, a load index value, based on the received signal obtained by the sensor device.

It is noted that within the meaning of physical movement is considered a static use of one or more muscles not necessarily giving rise to a physical movement of a body part as well as use of muscles resulting a physical movement of a body part. As such by bodily activity is preferable meant any change in a body, which will change the signal of the sensor and may give rise to an injury.

The period of time is preferable chosen such that the length of the signal is sufficient to extract strain characteristics. The time interval may therefore be as short as 5 min, potentially shorter, and as long as 24 hours, potentially longer.

Preferably, the load-index (abbreviated “LI” herein) value may be representative of the strain level of the user, such that a higher load-index value represents a higher chance of sustaining an strain injury, then a lower load-index value. The scale of the index may go from 0 to 10, wherein 0 represent the lowest possibility of strain and/or short and/or long-term injury to the body and 10 the highest. The user may then use this value to determine their present stain and change their behaviour in order to obtain a lower value. The load index, may also go from 0 to 100, and the scale may be seen as a relative scale, wherein the LI-value may be adapted to provide feedback based on a relative scale, wherein a larger number may be worse than a small number or vice versa.

A strain injury may be a muscular injury, damages to ligaments, tendons, cartilage, bones and other body parts that may be damaged due to strain.

It is noted that the invention does not provide any diagnosis in medical terms since it produces a load index value, and may give the user an appropriate warning and simple guidance on correction of the user's activity so as to avoid muscular and/or tissue damage. In order to reach a diagnosis, a medical practitioner will have to include other means such as e.g. x-ray images, age, gender, general physical conditions of the user etc. The invention is also based solely on non-invasive measurements methods.

The invention is used by a user (herein also referred to as “the user”).

The invention may be applied to fingers, wrists, elbows, shoulders, back, hips, neck, knees, ankles etc. The invention provides, at least potentially, the user to use his muscular activity in a manner where multiple repetitions of the same muscular activity is replaced by a diversity of muscular activities and/or bodily activities. Strength, duration and other parameters may be included in the method and system according to the invention.

The invention may be applied to any body-part, which experience strain, whereby strain is meant as a possibility of directly in a body part and/or indirectly in the same and/or another body part to sustain an injury. A bodily activity may therefore be seen as anything from a muscle group contracting to a joint position changing to general movement of body parts, which may indirectly be moved by a muscle not measured by the sensor device and no activity of the above mentioned.

In an example, the invention works by measuring, such as evaluating and/or recording, of bodily effect, such as muscular effect, by, but not limited to, e.g. non-invasive SEMG (surface electro myography) measurements, see FIG. 1. The invention converts this measuring to a signal that is, preferably, wirelessly sent to the user's smart device. The smart device can be a smart phone, tablet, computer, smart watch, but not limited to any of these. The signal is retrieved, transferred wirelessly, processed, and stored via an IT infrastructure and appropriate software. The software, preferably based on artificial intelligence, assesses the signal with respect to muscular power, number of repetitions, static work and time, but not limited to these. This assessment of the signal is processed and evaluated by the software into a near real-time feedback to the user, and the information is actively used to improve the basis for the wider feedback and stored for later potential statistical and medical research.

The sensor device may further be configured for being attached to a part of a user for sensing bodily activity(ies) in that part of a user during movement of said part and for providing a signal representative of the sensed bodily activity.

The invention may further comprise a communication device in signal communication with the sensor device, where the communication device comprises a detection unit, such that the detection unit physically forms part of the communication device.

The sensor used in connected with the invention may be of the type: SEMG, accelerator meter, gyro meter, near infra-red optical sensor, stretch sensor, force sensor, etc. The sensor type may preferable be chosen to fit to the sensed bodily activity and the location on the body, so as, to extract strain characteristics which may be used in the following calculation of the LI-value.

The signal from the sensor device may be processed to remove signal noise, such as by using RMS signal processing, restructuring of the data, compression of the signal, etc. This have the advantageous feature that the signal may have a cleaner base line for use in the calculation of the LI-value.

The load index value may be calculated using pre-defined research-based rules, based on extracted information concerning the strain-load characteristics leading to injuries, such as heavy load strain, high median strain, static strain, lack of rest etc., where the research-based rules may be obtained through research where the rules are based on a human evaluation of patterns leading to strain injuries. These research-based rules are advantageous as they may be defined parameters leading to injuries, however, there may be inconsistencies in the academic literature on what and how much strain leads to injuries.

The load-value index may solve this problem by, in an embodiment, developing a hierarchy-based system between the research-based rules and/or choosing the highest LI-value produced. As such, the invention may utilize many different research-based rules.

The research-based rules may come from both the academic research environment, as well as from other sources, such as from the data collected by the invention. The invention may be able to make its own research-based rules, based on general and specific data about the user.

The research-based rules may be based on the relative bodily activity level, which may be a relative muscular activity, defined as the ratio between the processed signal and the maximum voluntary amplitude, being above a threshold such as a 30% threshold, with/or without a signal length requirement. The signal length requirement may preferable be a percentage requirement of the total signal. This may have the advantage that the characteristics used to calculate the strain level may preferably be based on the relative strain level of the user. By maximum voluntary amplitude is preferable meant the maximum amplitude of the signal a user can make without externally exerted forces. The time threshold may normally be given by the research-based rules, such that the research may define a higher chance of strain damage if the potentially unhealthy relative bodily activity level has occurred for a certain percentage of the signal length.

A research-based rule may be set to be broken when a predefined percentage of the signal time length is over the research-based rule, such as 10% of the signal length is over a 30% threshold, giving the maximum LI-value. A research-based rule preferable may preferable be based on both time and strain thresholds, a signal exceeding both may be best represented as the maximum LI-value, eg. 10 in a 0-10 scale.

A research-based rule may be step-wise time and/or strain level aggregated, wherein the strain level may be the relative muscle activity level, such that an LI-value may be assigned to each time and/or strain level step in the research-based rule. This have the advantage that, since most research-based rules are threshold-based, they may not be able to capture the contribution of little strain over a small period, which may be below the time-requirement and/or strain requirement of the research-based rule. An aggregation of the rule may then be beneficial for monitoring little strain for small periods of time that occurs hourly, daily, weekly etc.

If two or more research-based rules calculates different LI-values, the highest LI-value may be chosen. As two or more research-based rules may come up with different LI-values, the highest LI-value may be chosen, as there may not have been developed a hierarchy between the rules. It is anticipated that a hierarchy system may be developed.

The LI-value may be calculated using a signal, originating from the sensor device and where the calculation of the LI-value comprises the steps of;
processing of the signal into a filtered signal, preferably into an RMS signal, and
calculation of a relative bodily activity level, defined as the ratio between the processed signal, preferable an RMS signal, and the maximum voluntary amplitude,
calculation of the relative time spent above a condition of a research-based rule,
use of the research-based rules to determine the LI-value.

The LI-value may also be calculated using statistical variables and/or functions related to the sensor signal.

The statistical variables, may be chosen from the mean of the sensor signal and/or the RMS, the standard deviation (STD) of the sensor signal and/or RMS signal, the power of the sensor signal and/or RMS, the time spent in a frequency category of a the frequency categorized signal, the relative difference in the maximum amplitude of the signal compared to the baseline level, the relative power in different frequency bands and/or the peak amplitude in a given frequency interval.

The load index may also be calculated based on a trained machine learning algorithm, wherein the input parameters for the learning is the pre-defined research-based rules, the user demographical data, strain patterns, such as repetitions, the statistical function and variables and characteristic originating from pain reports from the user. By demographical is preferable meant information relating to the age, occupation, hobbies, etc.

The load index-value may be updated on a regular basis, such as every 5 min, based on the signal period from the update. The updating of the LI-value may also be a larger or smaller than 5 min, such as 1 hour intervals or 2 min intervals.

The invention may further comprise a communication device that may be spatial apart from said detection unit, the communication device may be in signal communication with said sensor device and configured to
relay a signal received from said sensor device representative of a sensed bodily activity to the detection unit, and
receive from the detection unit a signal signaling the value of load index.

The detection unit may be configured for:
receiving signals from a plurality of sensor devices each signal being representative of a sensed bodily activity;
calculating, in each of said received signals, a load index value, and
storing said load index values in a database.

Although the invention has been detailed with reference to measurement of SEMG, the invention may also be based, either alone or in combination with SEMG measurement, on measurement of O2 and/or CO2 but not limited to this. Gyros and/or accelerometers may also by applied in connection with the present invention. The invention has the main advantageous that the measurements is non-invasive.

A list of recommendations may be produced, such as specific training and guidance for the user, based the LI-value history and/or the pain report and/or the user's demographical data. Such recommendations and guidance may be used to nudge the user toward healthy behaviour.

The invention may also prompt the use when a unhealthy LI-value is detected and alert the user, that the current activity is not ideal, therefore further nudging the user. The LI-value may therefore be considered a value that a user seeks to minimize through behavioural changes.

The sensor device may be configured for wireless transmitting said signal representative of the sensed bodily activity.

The detection unit may be configured for wireless receiving said signal representative of the sensed bodily activity and for wireless communicating the load index value.

The communication device may be configured for wireless communication with said sensor device and said detection unit.

The detection unit may be embodied in a user's smart device such as a smartphone, tablet, computer, smart watch.

The communication to the user wearing the sensor device that the load index value has been exceeded a predefined threshold may be in the form of visual, auditory or tactile perceptive information.

In a preferred embodiment the sensor device may comprise
a sleeve made from an elastic material, and/or another device configured for obtaining a signal representative of a position or movement of muscles, bone(s) and/or joint(s);
a device holding electronics
a socket attached to the sleeve or device, and configured for receiving the device holding electronics , the socket comprises electrical conductive connections for the connecting with device holding electronics,
a number of sensor strips arranged at the inside of the sleeve, the sensor strips are connected to the connections provided in the socket so as to provide a connection between the sensor strips and the device holding electronics.

In a second aspect, the invention relates to a method for monitoring and preferably evaluating physical movements of a user, the method comprising:
receiving a signal obtained by a sensor device (1) representative of a position or movement of muscles, bone(s) and/or joint(s),
calculating, in said received signal, a load index value,
preferably, communicate, e.g. to the user wearing the said sensor device (1), the load index value.

This aspect of the invention is particularly, but not exclusively, advantageous in that the present invention may be accomplished by a computer program product enabling a computer system to carry out the operations of the system of the first or the method according to the second aspect of the invention when down- or uploaded into the computer system. Such a computer program product may be provided on any kind of computer readable medium, or through a network.

The system and method according to the present invention may typically be computer implemented.

The individual aspects of the present invention may each be combined with any of the other aspects. These and other aspects of the invention will be apparent from the following description with reference to the described embodiments.

### BRIEF DESCRIPTION OF THE FIGURES

The different embodiments according to the invention will now be described in more detail with regard to the accompanying figures. The figures show one way of implementing the present invention and is not to be construed as being limiting to other possible embodiments falling within the scope of the attached claim set.

FIG. 1 schematically illustrates a first embodiment of a system according to the present invention, according to which movement of an elbow is sensed,

FIG. 2 schematically illustrates a second embodiment of a system according to the present invention,

FIG. 3 schematically illustrates a third embodiment of a system according to the present invention,

FIG. 4 is a flowchart detailing one embodiment of the load index value calculation method,

FIG. 5 illustrates an embodiment of a time table mapping of the research-based rule,

FIG. 6 is a photograph illustrating a user wearing a sensor device according to a preferred embodiment of the invention,

FIG. 7 is a photograph illustrating the sensor device of FIG. 6, in the figure the device holding electronics is shown detached from a socket,

FIG. 8 is a photograph illustrating the device holding electronics of FIGS. 6 and 7,

FIG. 9 is a photograph illustrating sensor device of FIG. 6 as seen from reverse side and

FIG. 10 is a flow diagram illustrating an embodiment of the invention delivering feedback to the user is shown.

### DETAILED DESCRIPTION OF AN EMBODIMENT

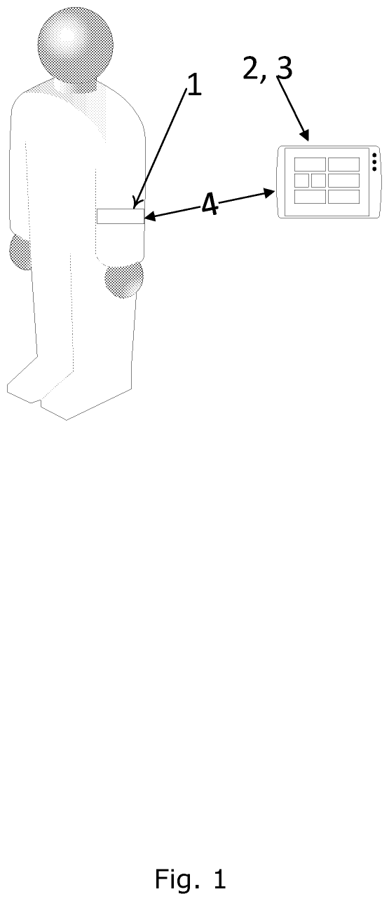

Reference is made to FIG. 1 schematically illustrating an embodiment of a system according to the present invention. The system is configured for monitoring and evaluating physical movements of a user or obtaining a position of a bone/joint. Such monitoring and evaluation may be carried out while the user is working, resting, thus in general monitoring physical movement of the user irrespectively of whether or not the user is activating his or hers muscles.

The system as illustrated comprises a detection unit 3 configured for receiving a signal obtained by a sensor device 1 representative of a sensed bodily activity.

Hereafter the embodiments referred to illustrates embodiments, wherein the sensor device senses a muscular activity. In other embodiments, the sensor might sense other bodily activities, such as joint position and/or bones. Furthermore, in the following embodiments muscular injuries are mentioned, but the invention also includes injuries due to strain of bodily activities, and the invention is not limited to only concern muscular injuries, but also includes strain injuries etc.

Such a detection unit may be a computer, smart phone or the like and the signal may be received via wireless communication, such as a WIFI or Bluetooth connection. The signal received is typically a time wise stream of data, where each data point represents muscular strain and/or load at a certain point in time.

The detection unit 3 is configured for calculating based on the signal obtained by the sensor device 1 over a defined time period, such as a 1 hour period, a load index (LI) value. The LI-value is based on the received signal obtained by the sensor device.

It has been shown that in the prevention of muscular injuries, an important factor to be considered is the total strain level of a muscular group. A muscular strain could be contributed by a muscular activity or a muscular load, such as flexing of the back or movement and use of the elbow. In the following muscular load, activity and strain are used interchangeable.

The defining feature of the LI-value is therefore that it is representative of the total muscular strain experience by the user. The LI-value can in one embodiment encompass a pain level factor from the user. The LI-value is based upon a measured period of the sensor signal and this period is typically around 1 hour, but can be any amount of time, such as 10 min or 3 hours, as long as the period is sufficiently long for extracting strain characteristics.

The LI-value is calculated by the detection device 1 as an identification of the muscular strain experience by the sensed muscle groups. The LI-value can go from 0 to 10, where 0 represent the lowest possibility of strain and/or short and/or long-term injury to the body and 10 the highest. The LI-value is calculated as one value over a sensed period, which will be detailed below. The sensor signal, such as an EMG signal, allows for the extraction of strain characteristics, such as sustained periods of high-level strain in the muscle based on the sensor signal.

As outlined, the detection unit 3 processes information received from a sensor device 1, and accordingly, the system may further comprise such sensor device 1. The sensor device 1 is typically configured for being attached to a part of a user for sensing muscular activity(s) in that part of the user during movement of said part and for providing a signal representative of the sensed muscular activity. Such sensor device may be a sensor measuring a mechanical response of muscle activity but other sensor types may be used in connection with the present invention, such as accelerator meter, gyro meter, near infra-red optical sensor, stretch sensor. The sensor device 1 further comprising—or is connected to—a transmitter, transmitting the sensed signal.

The signal from the sensor device 2 is to be received by the detection unit 3 and in some preferred embodiments, this data communication is handled by a communication device 2 forming part of the system. The communication device 2 is in signal communication 4 with said sensor device 1 for receiving data from the sensor device. Further, the communication device 2 may comprise detection unit 3, such as the detection unit 3 physically forms part of said communication device 2.

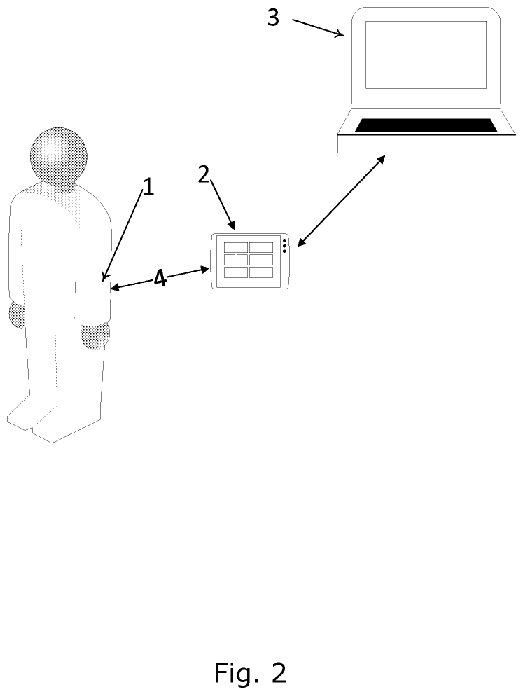

In another configuration of the system—see FIG. 2—wherein the system comprising a communication device 2, the communication device 2 is spatial apart from said detection unit 3. By spatial apart may typically mean that the detection unit 3 is at a different physical location, than the communication device, e.g. the detection communication device 2 may be a smart phone, and the detection unit 3 may be a centrally hosted server implementation.

In such embodiments, the communication device 2 is in signal communication 4 with the sensor device 1 and is configured to relay a signal received from sensor device 1 representative of a senses muscular activity to the detection unit 3, and receive from the detection unit 3, a signal signaling the value of the load index calculation.

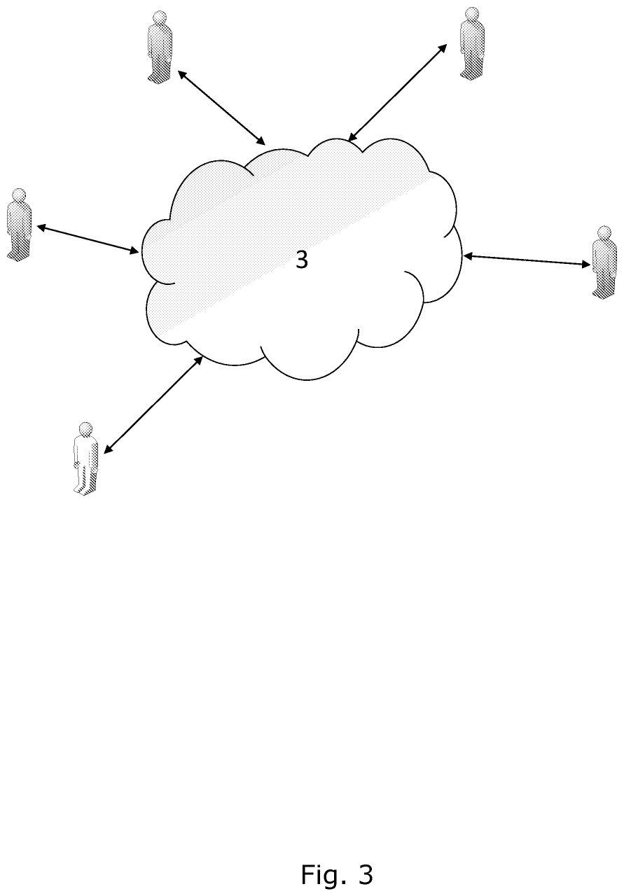

As illustrated in FIG. 3, another embodiment of the invention involves data from a plurality of users each wearing a sensor device 1. This is schematically illustrated in FIG. 3 in which the detection unit 3 is illustrated as a cloud communicating with sensor device 1 (uploading) and a communication device 2 (not shown) arranged in vicinity of each user, that is in position allowing the user to recognize information provided by the communication device 2. In such embodiments, the detection unit 3 is configured for receiving signals from a plurality of sensor devices 1, each signal being representative of a sensed muscular activity.

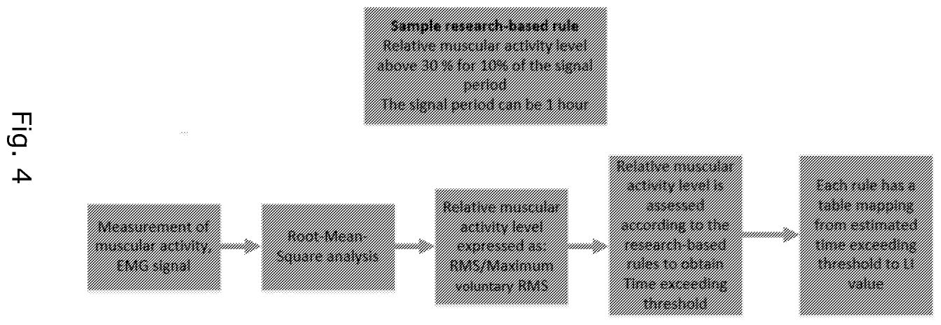

Referring to FIG. 4 a flow diagram detailing the steps involved, in accordance with an embodiment of the invention, in calculating the LI-value is shown. In a first step, the signal, received by the sensor device is processed to be able to extract information about the strain levels of the sensed muscle groups. The processing can be either a signal-processing step, wherein the signal noise is eliminated and/or a conversion of the signal into statistically functions and/or variables or a combination of both. The specific processing depends on the sensor type and the muscular groups being sensed and in some embodiment the research-based rules which will be detailed below. The used processing are known standard procedures in the field. The strain level in an embodiment is represented as a RMS processed sensor signal. The sensor signal will in the calculation be saved and used as a signal over a period of time, such as a 1 hour period.

The arrows in the flow diagram represents the direction of steps taken in the process.

In a preferred embodiment the statistical variables are chosen from the mean of the sensor signal and/or the RMS, the standard deviation (STD) of the sensor signal and/or RMS signal, the power of the sensor signal and/or RMS, the time spent in a frequency category of a the frequency categorized signal, the relative difference in the maximum amplitude of the signal compared to the baseline level, the relative power in different frequency bands and/or the peak amplitude in a given frequency interval.

If the sensor type is changed, the processing will in some circumstances change. For example, in a gyroscope sensor an orientation-processing step might be required.

In a second step the relative bodily activity level is calculated, which in this embodiment is a relative muscular activity, RMA, level. The relative muscle activity level being the ratio between the muscular strain level and the maximum voluntarily strain level, wherein the maximum voluntarily strain level represents an individual's maximum strain level under normal circumstances, where under normal circumstances is meant a user's ability to strain the muscle without outside exerted force In some embodiments, the maximum strain level is replaced by a different individual-based control variable. The important factor in the RMA-level is that it a relative value for the strain level for a specific user.

The maximum voluntary strain level is individually determined and is advantageous determined during a calibration step, undertaken during the set-up of the system. The relative muscle activity level can be expressed a percentage value.

The specific calculation of the RMA-value differs from sensor type to sensor type. In an EMG signal, which might be a sensor type used to sense muscular activity in the elbow, it is the ratio between the RMS and maximum RMS of the EMG-signal, wherein the maximum RMS-value represent the maximum voluntary strain level as described above. This normally produces a value between 0 and 100%, but can in some situations exceed 100%, such as when a muscle is torn or a bone is broken. In such a case, the value of 100% is used. The RMA can be expressed as

$$RM A = \frac{proc essed sensor signal}{maximum voluntary strain level} * 100 \% .$$

In a third step, the RMA-value is used to calculate the LI-value. In one embodiment, the LI is based on scientific research-based rules, which utilizes the signal obtained by the sensor device. A research-based rule is a rule based on research concerning factors leading to strain injuries. These rules are in the majority of cases independent of subjective factors, such as age, occupation etc., only being based upon strain levels.

Such research-based rules can be based on threshold rules, and can be expression for heavy load strain, high median strain, static strain and lack of rest (restitution), which is scientifically proven to lead to an injury if not corrected or treated. These research-based rules changes for the specific muscular groups and/or sensor types. The research-based rules are normally characterized in that they encompasses both a strain level requirement and a time requirement.

An example of a research-based rule could be that the RMA-value is over a specified RMA-value, such as 30% percent, for a specified amount of time, such as 10% of the signal length. A research-based rule is said to be broken when both conditions are satisfied, such that the above rule is broken if the RMA-value is over 30% for 12% of the time. A broken research-based rule is associated with an LI-value of 10 or the highest value on the scale.

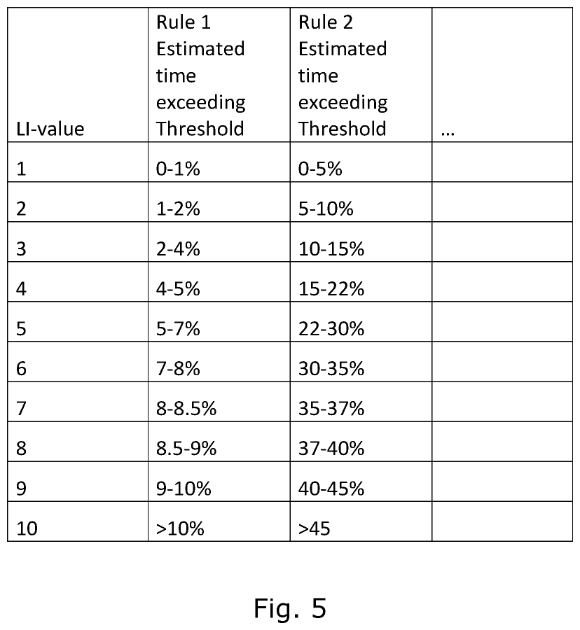

Referring to FIG. 5, the research-based rules can be further aggregated into percentage breakage of the rule, wherein by breakage is meant a partial fulfilment of the research-based strain level and/or time requirements. Such an aggregation occur by table mapping the research-based rules into estimated time exceeding thresholds, wherein each entity in the table has a different LI value associated.

The table shown in FIG. 5 is time aggregated, such that the time interval the RMA-value is over 30% gives a different LI-value depending on the length of the time interval until the rule is broken, as a broken research-based rule is associated with an LI-value of 10. The research-based rules can also be aggregated by other ways, such as in the strain level requirement.

For example, as seen in FIG. 5, if the relative muscle percentage is over 30% for only 5% of the time, wherein the research-based rule is only broken when it is over 30% for 10% of the time, an LI-value of 6 could be assign to the period. A mathematical expression for a research-based aggregated rule can be

$$LI = Tabel [ \frac{{\int }_{0}^{T} dx RMA ( if research parameter i s true )}{{\int }_{0}^{T} dx RMA ( x )} ] .$$

where T is the signal period. In FIG. 5 an example of a table-mapped research-based rule to LI-values is shown. The table mapping could be non-linear and based on empirical findings.

The different research-based rules are in one embodiment independent from each other, such that the highest LI-value across the different research-based rules is chosen as the final value. It is anticipated that a hierarchical system could be developed.

The research-based rules can also be further segmented into rules for healthy, previous injured and injured individuals.

Furthermore, the LI can advantageous be calculated using a trained machine-learning algorithm with/or without the research-based rules. Such a machine learning system would be trained to detect characteristics leading up to a pain report, which is not anticipated by the research-based rule, such characteristics could be repetition of movements, energy content in the time and/or frequency domain and identification of patterns leading up to a pain report. The machine-learning algorithm could be trained using user-feedback and initial data obtained by using the research-based rules, as well as the pain and strain level indications predicted by the research-based rule.

The training of the machine-learning algorithm could in an embodiment be accomplish by utilizing the research-based parameters to obtain a first indication of the relevant parameters for the training and to narrow in the training area of the machine learning algorithm. The training material can be accomplish by inter-correlating extract features from the sensor, e.g. from a Gyro Accelerometer or EMG signal, as well as user related parameters, such as e.g. age, gender, injury status etc. and pain reports reported by the users, as well as parameters in the signal representing muscular strain.

The machine learning could in an embodiment be trained using the research-based rules as a foundation for the algorithm and as the repository of pain report and associated history is expanded, the machine will shift to relying on this instead of the research-based rules. Such a transitions can be said to be going from an objective research-based training to a subjective empirically based training of the algorithm.

The strain parameters representing muscular strain comprises both parameters from the research-based rules, as well as additional parameters such as parameters identifying the energy content both in time and frequency domain and patterns leading up to a pain report—e.g. correlation with a pre-identified signal pattern.

The machine-learning would therefore be able, by using the user related parameters, strain parameters and pain report, to add new analysis dimensions to the LI-value, and in greater detail specifically tailor the LI-value to the individual user. As such, the main advantage of the machine-learning version is that the load index is more nuance and would eventually be able to take over from the research-based rules.

The machine-learning algorithm has in addition the advantage that it can intercorrelate patterns that leads to pain, by analysing the pain report submitted by the user and comparing the pain level with the sensor data in the analysis period. The machine-learning algorithm can also adjust the scale of the LI for the specific user.

The statistical variables used as parameters for the machine learning and/or research-based rules, if the sensor signal is an EMG measurement, could be chosen from the mean of the SEMG and/or the RMS, the standard deviation (STD) of the EMG and/or RMS signal, the power of the EMG and/or RMS, the time spent in a frequency category of a the frequency categorized signal, the relative difference in the maximum amplitude of the signal compared to the baseline level, the relative power in different frequency bands and/or the peak amplitude in a given frequency interval.

The LI-value can in an embodiment be continuously updated every time a specific time interval has occurred, such as every 5 min, but the time interval can be larger or smaller than this. The LI value can in an embodiment be used to give recommendations to the user. Such recommendations can be based on a single LI-value or a history of LI-values. The recommendations could be general guidelines or modified to the specific user based upon the users characteristics as provided by the user.

The invention may be characterised by being independent of professionals instructing the user, and can be used by the user without instruction, which will save time and expensive consultation. The invention is intuitive in use and does not require extra equipment as previous solutions, making it more accessible to the user, so it can be integrated into the user's everyday life. The user is therefore expected to use the invention more frequently and the use will have a more lasting effect. The invention is based on measurements of several parameters that are collected from the sensors, which are sent wirelessly to the user's smart device, where the signal is processed through algorithms to ultimately give the user feedback on, for example, a potential overload of a muscle, or feedback to corrective measure.

The invention can be implemented by means of hardware, software, firmware or any combination of these. The invention or some of the features thereof can also be implemented as software running on one or more data processors and/or digital signal processors.

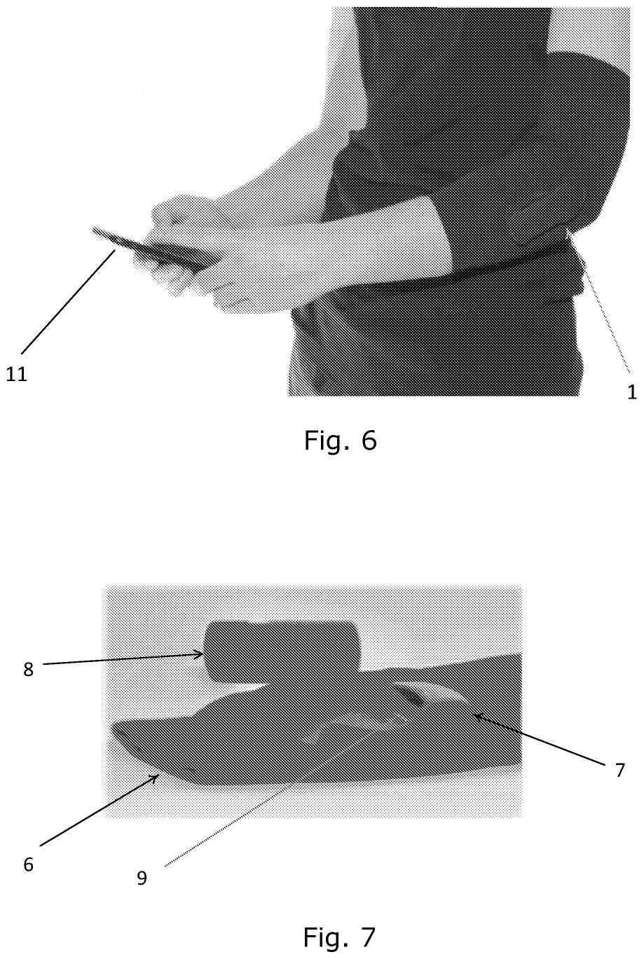

Reference is now made to FIGS. 6-9, whereof FIG. 6 is a photograph illustrating a user wearing a sensor device 1 according to a preferred embodiment of the invention. The sensor device comprising a tubular sleeve 6 made from an elastic material, such as an elastic fabric allowing it to fit firmly on the user's arm as illustrated and allowing a user to pass his hand and arm through interior of sleeve when the sensor device 1 is to be arranged on the arm. As also illustrated in FIG. 6, the user carries a smartphone which may be configured (as other-wise disclosed herein) to operate as a communication device and/or detection device.

Although the sensor device 1 is illustrated as being used on an arm and elbow, the same principle may and in some instances, the same device 1, may be used on different body parts. Such body parts wherein the device 1, can advantageous be used could be on the back, the neck, the knee including the kneecaps, the thigh, etc.

With reference to FIG. 7, which is a photograph illustrating the sensor device 1 of FIG. 6. As illustrated, the sensor device comprises a device holding electronics 8. Such electronics are preferably the electronic components used for converting the sensed signal into data and transmitting such data to the communication device and/or the detection unit 3. The electronic components may also include data memory, data processor, battery(ies), accelerometer and/or gyros and/or other sensor which measure muscle activities which is of decisive importance for potential harmful behaviour. In FIG. 7 the device holding electronics 8 is shown detached from a socket 7 which is attached to the sleeve 6. The socket also comprises conductive (typical electrically conductive) connections 9 for connecting with the device holding electronics 8, the purpose of which will be disclosed on connection with the 7. The socket 7 is adapted to receive the device holding electronics 8 in a firm fit preventing the device 8 to fall out of the socket 7 during the user wearing the sensor device 1. The device holding electronics 8 comprising connections (not illustrated) mating the connections 9 once the device 8 is arranged in the socket to allowing electrical connections between the device holding electronics 8 and the connections 9.

By use of a socket 7, such a socket may be applied to different shaped sleeves 6, whereby different sleeves 6 may be used to accommodate the same device holding electronics 8.

In FIG. 8 the device holding electronics of FIGS. 6 and 7 is illustrated as a photograph. FIG. 8 illustrates inter alia that the device holding electronics may comprises a USB connection 12 allowing access to data stored in the device, programming of data processors (if present) and/or charging rechargeable battery(ies), if present.

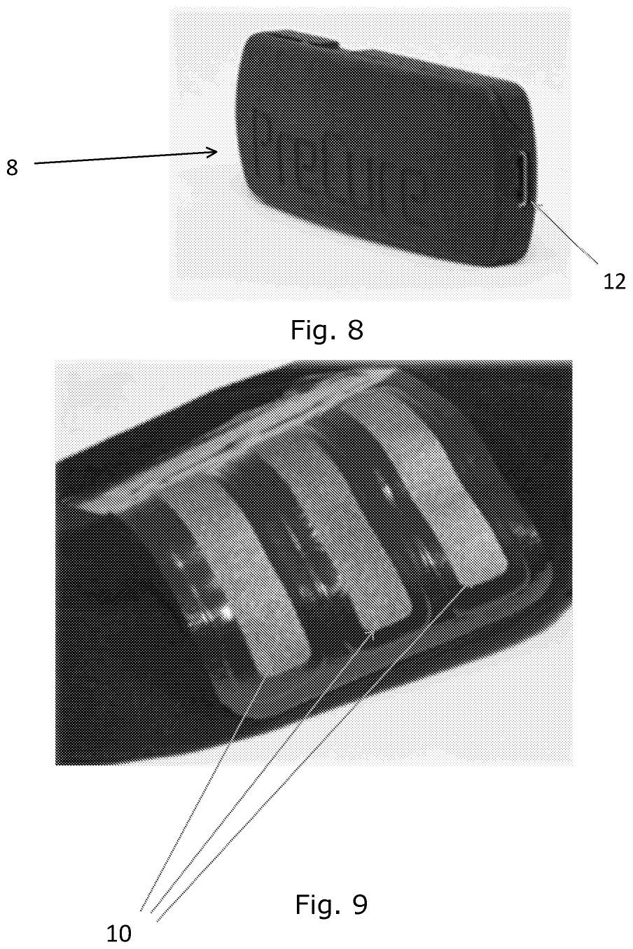

Reverting now to FIG. 9 which is a photograph illustrating the sensor device 1 of FIG. 6 as seen from reverse side. By reverse side means that the sleeve 6 has been turned inside-out revealing the inner surface of the sleeve 6 which during use abut the skin of the user. As illustrated in FIG. 9, a number of sensor strips 10, such electrical sensor strips is arranged at the inside of the sleeve 6; in the embodiment of FIG. 9, three such sensor strips 10 are arranged. The sensor strips 10 may be arranged parallel to each other. The sensor strips 10 are connected to the connections 9 provided in the socket 7 so as to provide a connection between the sensor strips 10 and the device holding electronics 8.

In other embodiments, the sensor is fastened to other parts of the body and measure different aspect to calculate the LI-value. As the LI-value is representative of relative load on a specific muscle group, multiple sensors and measurements techniques can be utilized. For example, if the sensor is placed on a person's back, the sensor could be of the type of a near-infrared optical sensor measuring blood oxygen saturation values and/or acidification. In such a situation the measured force used, such as when the spine is bent, can be indirectly measured by looking at the blood oxygen saturation values and/or acidification.

The position and rotation are also important in the prevention of injuries, and these can be measured by the use of accelerator meter and gyro meter sensors, and can be utilized at different positions on the body. The bending of the spine can be measured by use of a stretch sensor, measuring the arching of the back.

These parameters is also relevant for other parts of the body. The sensors might need to be adapted to the specific body part, as requirements such as position, sweat, body curvature etc. needs to be overcome. The general principle is described for the case of the elbow but the specific sleeve or sensor-holding object will change accordingly.

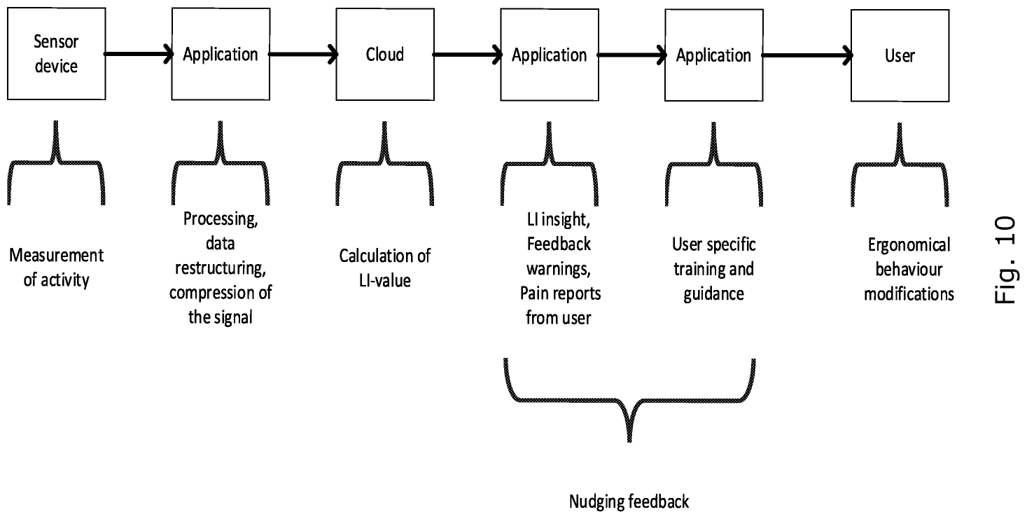

Now referring to FIG. 10, a flow diagram over an embodiment of the invention delivering feedback to the user is shown. The arrows represents the flow of operation. As the LI-value is representative of strain in a user of the system, a list of recommendation can be produced nudging the user to reconsider their behaviour. By nudging is meant behavioural modifying information and/or recommendations to the user.

The sensor device 1 will measure the activity of the user for a defined period of time, and send the signal data to an application, which will processed the data. This data processing could be, as detailed above a RMS signal processing, restructuring of the data, compression of the signal, etc. This data can, in an embodiment, be sent to a cloud, where the LI-value will be calculated.

The calculation of the LI-value has been detailed in previous in the application but may be based on research-based rules or a machine-learning algorithm. The cloud will, in an embodiment, calculate the LI-value and transfer the LI-value back to the application. The application will, based on the LI-value or history of LI-values, produce an insight to the LI-value. This insight may include recommendations, feedback warnings and general information relating to bodily activity. The application may also collect pain reports from the user in order to produce the insight and use the pain report in the analysis of the LI-values and/or the training of the machine-learning algorithm.

The application will, based on the analysis done in the previous step, give the user specific training and guidance in order to lower the chance of strain injuries. The user will therefore have optimal information to make ergonomically behaviour modifications, thereby lowering the chance of sustaining strain injuries.

The application will therefore nudge the user away from unhealthy situations by providing suggestions on training and guidance, as well as warnings when the LI-value becomes too high and an unhealthy activity might be performed or is being performed. A user will be able to analyse their daily activity and compared these activities to the LI-values, and make appropriate adjustments so as to keep the LI-value as low as possible. This may help prevent or alleviate strain injuries.

The invention can be implemented by means of hardware, software, firmware or any combination of these. The invention or some of the features thereof can also be implemented as software running on one or more data processors and/or digital signal processors.

The individual elements of an embodiment of the invention may be physically, functionally and logically implemented in any suitable way such as in a single unit, in a plurality of units or as part of separate functional units. The invention may be implemented in a single unit, or be both physically and functionally distributed between different units and processors.

Although the present invention has been described in connection with the specified embodiments, it should not be construed as being in any way limited to the presented examples. The scope of the present invention is to be interpreted in the light of the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

1. A system for monitoring the movements of a user, the system comprising:
a detection unit (3) configured for
receiving a signal obtained by a sensor device (1) representative of a sensed bodily activity, said bodily activity optionally comprising a position or movement of muscles, bone(s) and/or joint(s),
calculate based on the signal obtained by the sensor device (1) over a period of time, optionally a 1 hour signal period, a load index (LI-) value based on the received signal obtained by the sensor device (1).

2. The system according to claim 1, the system being configured for communicating the value of the load index, the communicating optionally being to the user wearing the said sensor device (1).

3. The system according to claim 1, said sensor device (1) being configured for being attached to a part of a user for sensing one or more bodily activities in that part of a user during movement of said part and for providing a signal representative of the sensed bodily activity.

4. The system according to claim 1, further comprising a communication device (2) in signal communication (4) with said sensor device (1), said communication device (2) comprising said detection unit (3), optionally such that said detection unit (3) physically forms part of said communication device (2).

5. The system according to claim 1, wherein the sensor is characterized as a SEMG sensor, accelerator meter, gyro meter, near infra-red optical sensor, stretch sensor, or force sensor.

6. The system according to claim 1, wherein the signal from the sensor device (1) is processed to remove signal noise, optionally by using RMS signal processing.

7. The system according to claim 1, wherein the load index value is calculated using one or more pre-defined research-based rules, based on extracted information concerning the strain-load characteristics leading to injuries, wherein the extracted information optionally includes heavy load strain, high median strain, static strain, lack of rest, or any combination thereof.

8. The system according to claim 7, wherein the research-based rules are based on a relative bodily activity level that is defined as the ratio between the processed signal and the maximum voluntary amplitude being above a threshold, with/or without a signal length requirement, the threshold optionally being a 30% threshold.

9. A system according to claim 7, wherein a research-based rule is set to be broken when a predefined percentage of the signal time length is over the research-based rule giving the maximum LI-value, optionally wherein 10% of the signal length is over a 30% threshold.

10. A system according to claim 8 wherein in a research-based rule is step-wise time and/or strain level aggregated, wherein the strain level is the relative bodily activity level, so that an LI-value is assigned to each time and/or strain level step in the research-based rule.

11. A system according to claim 7 wherein if two or more research-based rules yield different LI-values, the highest LI-value is chosen.

12. The system according to claim 1, the system being configured to calculate the LI-value using a signal originating from the sensor device (1) and wherein the calculation of the LI-value comprises the steps of:
processing of the signal into a filtered signal, preferably into an RMS signal,
calculation of a relative bodily activity level, defined as the ratio between the processed signal, preferable an RMS signal, and the maximum voluntary amplitude, and
calculation of the relative time spent above a condition of a research-based rule,
use of the research-based rules to determine the LI-value.

13. The system according to claim 1, wherein the LI-value is calculated using statistical variables and/or functions related to the signal obtained by the sensor device.

14. The system according to claim 13, where the statistical variables are one or more of the mean of the sensor signal and/or the RMS, the standard deviation (STD) of the sensor signal and/or RMS signal, the power of the sensor signal and/or RMS, the time spent in a frequency category of a the frequency categorized signal, the relative difference in the maximum amplitude of the signal compared to the baseline level, the relative power in different frequency bands and/or the peak amplitude in a given frequency interval.

15. The system according to claim 7, wherein the LI-value is calculated based on a trained machine learning algorithm, wherein the input parameters for the learning are the pre-defined research-based rules, the user demographical data, strain patterns, the statistical function and variables and characteristic originating from pain reports from the user.

16. The system according to claim 1, wherein the load index-value is updated on a regular basis, optionally every 5 minutes, based on the signal period from the update.

17. The system according to claim 1, wherein the load index-values are stored in a database.

18. The system according to claim 1, wherein the system is configured to provide a list of recommendations based on a LI-value history and/or a pain report and/or the user's demographical data, the list of recommendations optionally comprising training and guidance for the user.

19. The system according to claim 1, further comprising a communication device (2) spatially apart from said detection unit (3), the communication device (2) being in signal communication (4) with said sensor device (1) and the communication device (2) being configured to
relay a signal received from said sensor device (1) representative of a senses bodily activity to the detection unit (3), and
receive from the detection unit (3) a signal signaling the value of load index.

20. The system according to claim 1, wherein the detection unit (3) is configured for:
receiving signals from a plurality of sensor devices (1) each signal being representative of a sensed bodily activity;
calculating, in each of said received signals, a load index value, and
storing said load index values in a database.

21. The system according to claim 1, wherein the sensor device (1) is configured for wirelessly transmitting said signal representative of sensed bodily activity.

22. The system according to claim 1, wherein the detection unit (3) is configured for wireless receiving said signal representative of the sensed bodily activity and for wireless communicating the load index value.

23. The system according to claim 4, wherein the communication device (2) is configured for wireless communication with said sensor device (1) and said detection unit (3).

24. The system according to claim 1, wherein the detection unit (3) is embodied comprised in a user's smart device, said smart device optionally comprising a smartphone, tablet, computer, or smart watch.

25. The system according to claim 2, wherein the system is configured to communicate to the user wearing the sensor device (1) that the load index value has exceeded a predefined threshold, and the communicating is in the form of visual, auditory or tactile perceptive information.

26. The system according to claim 1, wherein the sensor device (1) comprises
a sleeve (6) made from an elastic material, and/or another device configured for obtaining a signal representative of position or movement of muscles, bone(s) and/or joint(s);
a device holding electronics (8)
a socket (7) attached to the sleeve (6) or device, and configured for receiving the device holding electronics (8), the socket comprises electrical conductive connections (9) for the connecting with device holding electronics (8),
one or more sensor strips (10) arranged at the inside of the sleeve (6), the one or more sensor strips (10) being connected to the connections (9) provided in the socket (7) so as to provide a connection between the one or more sensor strips (10) and the device holding electronics (8).

27. A method for monitoring and optionally evaluating physical movements of a user, the method comprising:
receiving a signal obtained by a sensor device (1) representative of a position or movement of muscles, bone(s) and/or joint(s),
calculating, in said received signal, a load index value,
optionally communicating to a user wearing the said sensor device (1), the load index value.
